(19) United States
(12) Patent Application Publication
(10) Pub. No.: US 2010/0215541 A1
(43) Pub. Date: Aug. 26, 2010

(54) Device and method for producing high power microwave plasma

(76) Inventor: Ralf Spitzl, Troisdorf (DE)

Correspondence Address: D. PETER HOCHBERG CO. L.P.A., 1940 EAST 6TH STREET, CLEVELAND, OH, 44114, US

(21) Appl. No.: 12/311,838

(22) PCT Filed: Oct. 11, 2007
(86) PCT No.: PCT/EP2007/008838

§ 371 Date: Jul. 20, 2009

(30) Foreign Application Priority Data
Oct. 16, 2006 (DE) 10 2006 048 815.6

(51) Int. Cl. A61L 2/14; H05H 1/30; B01J 19/12; C23F 1/08; C23C 16/513; C23F 1/04

(52) U.S. Cl. 422/28; 204/164; 422/186.04; 156/345.41; 427/575; 216/69; 422/186.03; 156/345.4

(57) ABSTRACT

A device for producing high power microwave plasmas. The device comprises at least one microwave feed that is surrounded by at least one dielectric tube. A dielectric fluid flows through the space between the microwave feed and the outer dielectric tube. The dielectric fluid has a small dielectric loss factor tan δ in the region of between 10⁻² to 10⁻⁷. A fluid cools at least the outer dielectric tube.

### CROSS-REFERENCE TO RELATED APPLICATIONS

This application is a National Stage application of International Application No. PCT/EP2007/008838, filed on Oct. 11, 2007, which claims priority of German application number 10 2006 048 815.6, filed on Oct. 16, 2006, both of which are incorporated herein by reference in their entireties.

### BACKGROUND OF THE INVENTION

1. Field of the Invention

The present invention relates to a method for generating microwave plasmas of high plasma density in a device that comprises at least one microwave feed that is surrounded by at least one dielectric tube.

2. Description of the Prior Art

Devices for generating microwave plasmas are being used in the plasma treatment of workpieces and gases. Plasma treatment is used, for example, for coating, cleaning, modifying and etching workpieces, for treating medical implants, for treating textiles, for sterilisation, for light generation, preferably in the infrared to ultraviolet spectral range, for converting gases or for gas synthesis, as well as in waste gas purification technology. To this end, the workpiece or gas to be treated is brought into contact with the plasma or the microwave radiation.

The geometry of the workpieces to be treated ranges from flat substrates, fibres and webs, to any configuration of shaped articles.

Any known gas can be used as the process gas. The most important process gases are inert gases, fluorine-containing and chlorine-containing gases, hydrocarbons, furans, dioxins, hydrogen sulfides, oxygen, hydrogen, nitrogen, tetrafluoromethane, sulfur hexafluoride, air, water, and mixtures thereof. In the purification of waste gases by means of microwave-induced plasma, the process gas consists of all kinds of waste gases, especially carbon monoxide, hydrocarbons, nitrogen oxides, aldehydes and sulfur oxides. However, these gases can be used as process gases for other applications as well.

Devices that generate microwave plasmas have been described in the documents WO 98/59359 A1, DE 198 480 22 A1 and DE 195 032 05 C1.

The above-listed documents have in common that they describe a microwave antenna in the interior of a dielectric tube. If microwaves are generated in the interior of such a tube, surface waves will form along the external side of that tube. In a process gas which is under low pressure, these surface waves produce a linear elongate plasma. Typical low pressures are 0.1 mbar-10 mbar. The volume in the interior of the dielectric tube is typically under ambient pressure (generally normal pressure; approx. 1013 mbar). In some embodiments a cooling gas flow passing through the tube is used to cool the dielectric tube.

To feed the microwaves, hollow waveguides and coaxial conductors are used, inter alia, while antennas and slots, among others, are used as the coupling points in the wall of the plasma chamber. Feeds of this kind for microwaves and coupling points are described, for example, in DE 423 59 14 and WO 98/59359 A1.

The microwave frequencies employed for generating the plasma are preferably in the range from 800 MHz to 2.5 GHz, more preferably in the range from 800 MHz to 950 MHz and 2.0-2.5 GHz, but the microwave frequency may lie in the entire range from 10 MHz up to several 100 GHz.

DE 198 480 22 A1 and DE 195 032 05 C1 describe devices for the production of plasma in a vacuum chamber by means of electromagnetic alternating fields, comprising a conductor that extends, within a tube of insulating material, into the vacuum chamber, with the insulating tube being held at both ends by walls of the vacuum chamber and being sealed with respect to the walls at its outer surface. The ends of the conductor are connected to a generator for generating the electromagnetic alternating fields.

A device for producing homogenous microwave plasmas according to WO 98/59359 A1 enables the generation of particularly homogeneous plasmas of great length, even at higher process pressures, as a result of the homogeneous input coupling.

The possible applications of the above-mentioned plasma sources are limited by the high energy release of the plasma to the dielectric tube. This energy release may result in an excessive heating of the tube and ultimately lead to the destruction thereof. For that reason, these sources are typically operated at microwave powers of about 1-2 kW at a correspondingly low pressure (approximately 0.1-0.5 mbar). The process pressures can also be 1 mbar-100 mbar, but only under certain conditions and at a correspondingly low power, in order not to destroy the tube.

With the above-mentioned devices, typical plasma lengths of 0.5-1.5 m can be achieved. With plasmas of almost 100% argon it is possible to achieve greater lengths, but such plasmas are of little technical importance.

Another problem with such plasma sources lies in the channeling of the process gas, especially at higher process gas pressures (above 1 mbar). The reason for this is that with increasing radial distance from the dielectric tube, the plasma density decreases strongly. This makes it more difficult to supply new process gas to the areas of high charge carrier density. In addition, at higher process pressures, the thermal power dissipated to the dielectric tube increases.

However, higher process gases are preferred since they frequently result in a clear, tenfold to hundredfold, increase in the process velocity.

### SUMMARY OF THE PRESENT INVENTION

It is the object of the present invention to prevent or reduce the above-mentioned disadvantages of excessive heating of the dielectric tube and thereby to achieve an increase in the power of the plasma sources.

This object is achieved by a method according to the present invention. In a device for generating microwave plasmas, which comprises at least one microwave feed surrounded by at least one dielectric tube, a dielectric fluid is conducted through the space between the microwave feed and the dielectric tube. The dielectric fluid, which has a low dielectric loss factor tan δ in the range of from 10⁻² to 10⁻⁷, flows through the space between the microwave feed and the dielectric tube.

By means of the above method it is possible to cool, in an advantageous manner, the dielectric tube by conducting the fluid through the above-described arrangement of tubes.

The device and the method will be described in the following.

Suitable microwave feeds are known to those skilled in the art. Generally, a microwave feed consists of a structure which is able to emit microwaves into the environment. Structures that emit microwaves are known to those skilled in the art and can be realised by means of all known microwave antennae and resonators comprising coupling points for coupling the microwave radiation into a space. For the above-described device, cavity resonators, bar antennas, slot antennas, helix antennas and omnidirectional antennas are preferred. Coaxial resonators are especially preferred.

In service, the microwave feed is connected via microwave feed lines (hollow waveguides or coaxial conductors) to a microwave generator (e.g. klystron or magnetron). To control the properties of the microwaves and to protect the elements, it is furthermore possible to introduce circulators, insulators, tuning elements (e.g. 3-pin tuners or E/H tuners) as well as mode converters (e.g. rectangular and coaxial conductors) in the microwave supply.

The dielectric tubes are preferably elongate. This means that the tube diameter: tube length ratio is between 1:1 and 1:1000, and preferably 1:10 to 1:100. The two tubes may be equally long or be different in length. Furthermore, the tubes are preferably straight, but they may also be of a curved shape or have angles along their longitudinal axis.

The cross-sectional surface of the tubes is preferably circular, but generally any desired surface shapes are possible. Examples of other surface shapes are ellipses and polygons.

The elongate shape of the tubes produces an elongate plasma. An advantage of elongate plasmas is that by moving the plasma device relative to a flat workpiece it is possible to treat large surfaces within a short time.

The dielectric tubes should, at the given microwave frequency, have a low dielectric loss factor tan δ for the microwave wavelength used. Low dielectric loss factors tan δ are in the range from 10⁻² to 10⁻⁷.

Suitable dielectric materials for the dielectric tubes are metal oxides, semimetal oxides, ceramics, plastics, and composite materials of these substances. Particularly preferred are dielectric tubes made of silica glass or aluminium oxide with dielectric loss factors tan δ in the range from 10⁻³ to 10⁻⁴. The dielectric tubes here may be made of the same material or of different materials.

According to one particular embodiment, the dielectric tubes are closed at their end faces by walls.

A gas-tight or vacuum-tight connection between the tubes and the walls is advantageous. Connections between two workpieces are known to those skilled in the art and may, for example, be glued, welded, clamped or screwed connections. The tightness of the connection may range from gas-tight to vacuum-tight, with vacuum-tight meaning, depending on the working environment, tightness in a rough vacuum (300-1 hPa), fine vacuum (1-10⁻³ hPa), high vacuum (10⁻³-10⁻⁷ hPa) or ultrahigh vacuum (10⁻⁷-10⁻¹² hPa). Generally, the term “vacuum-tight” here refers to tightness in a rough or fine vacuum.

The walls may be provided with passages, through which a fluid can be conducted. The size and shape of the passages can be chosen at will. Depending on the application, each wall may contain at least one passage. In a preferred embodiment, there are no passages in the region that is covered by the face end of the inner dielectric tubes.

Via these passages, the fluid can be conducted into the space between the outer dielectric tube and the inner dielectric tube and it can also be discharged via these passages. Another possibility consists in the feeding and discharge, respectively, of the dielectric liquid via passages in the microwave feed, on the one hand, and at least one of the passages in the walls, on the other hand. The pressure of the fluid may be above, below or equal to the atmospheric pressure.

The flow velocity and the flow behaviour (laminar or turbulent) of the dielectric fluid flowing through the dielectric tube is to be chosen such that the fluid has good contact with the boundary of the dielectric tube and that, in addition, where a liquid fluid is used, there does not occur any evaporation of the dielectric liquid. How the flow velocity and flow behaviour can be controlled by means of pressure and by means of the shape and size of the passages is known to those skilled in the art.

Preferably, a dielectric liquid is used as the dielectric fluid. Since liquids generally have a much higher specific thermal coefficient than gases, cooling of the dielectric tube by means of a dielectric liquid is much more effective than gas cooling, as is described in DE 195 032 05 C1.

However, cooling of the dielectric tube by means of a liquid cannot be realised in an easy fashion since the energy input of the microwaves to the liquid results in the heating of the latter. Any additional heating of the dielectric liquid will decrease the cooling effect on the dielectric tube. This decrease in the cooling performance can also, if the microwave absorption by the liquid is high, lead to a negative cooling performance, which corresponds to an additional heating of the dielectric tube by the cooling liquid.

To keep the heating of the dielectric liquid by the microwaves as low as possible, the dielectric liquid must, at the wavelength of the microwaves, have a low dielectric loss factor tan δ in the range of 10⁻² to 10⁻⁷. This prevents a microwave power input into the fluid medium or reduces said input to an acceptable degree.

An example of such a dielectric liquid is an insulating oil that has a low dielectric loss factor. Insulating oils are, for instance, mineral oils, olefins (e.g. poly-alpha-olefin) or silicone oils (e.g. COOLANOL® or dimethyl polysiloxane). Hexadimethylsiloxane is preferred as the dielectric liquid.

By means of this fluid cooling of the outer dielectric tube, it is possible to reduce the heating of the outer dielectric tube. This enables higher microwave powers which, in turn, lead to an increase in the concentration of the plasma at the outside of the outer dielectric tube. In addition, the cooling enables a higher process pressure than in uncooled plasma generators.

Another embodiment of the device is a double-tube arrangement. Here, a dielectric inner tube is inserted between the microwave feed and the dielectric tube.

In this embodiment, the dielectric fluid can be conducted between the two tubes (see FIG. 2).

By contrast to the gas cooling according to DE 195 032 05, where the cooling gas is in contact with the microwave feed, in the present embodiment the contact between the fluid and the microwave feed is prevented by the double-tube arrangement, thereby excluding any possibility of the fluid reacting with the microwave feed. Furthermore, this separation of fluid and microwave feed greatly facilitates the maintenance of the microwave feed.

In order to further reduce the microwave power requirement for the above-mentioned plasma sources, according to another preferred embodiment it is possible for a metallic jacket to be applied around the outer dielectric tube, said jacket partially covering the tube. This metallic jacket here acts as a microwave shield and may be made, for example, of a metallic tube, a bent sheet metal, a metal foil, or even a metallic layer, and may be plugged or electroplated thereon, or applied thereon in another way. Such metallic microwave shields are able to limit the angular range in which the generation of the plasma takes place as desired (e.g. 90°, 180° or 270°) and thereby reduce the power requirement accordingly.

Especially in the case of the embodiment of the devices for generating microwave plasmas which comprises a metal jacket, it is possible to treat broad material webs with a plasma at a low power loss. The jacket shields that region of the space in the device which does not face the workpiece, and there is generated only a narrow plasma strip between the workpiece and the device, over the entire width of the workpiece.

All of the above-described devices for plasma generation, during operation, form a plasma at the outside of the dielectric tube. In a normal case, the device will be operated in the interior of a space, a plasma chamber. This plasma chamber may have various shapes and apertures and serve various functions, depending on the operating mode. For example, the plasma chamber may contain the workpiece to be processed and the process gas (direct plasma process), or process gases and openings for plasma discharge (remote plasma process, waste gas purification).

### BRIEF DESCRIPTION OF THE DRAWINGS

In the following, the invention will be explained, by way of example, by means of the embodiments which are schematically represented in the drawings.

FIG. 1 is a cross-sectional and a longitudinal-sectional view of the device according to the present invention.

FIG. 2 is a cross-sectional and a longitudinal-sectional view of another embodiment of the device according to the present invention, comprising a double-tube arrangement.

FIG. 3A is a cross-sectional view of one embodiment of the present invention comprising a metallic jacket.

FIG. 3B is a cross-sectional view of another embodiment of the present invention comprising a metallic jacket.

FIG. 4 is a longitudinal-sectional drawing of the device according to the present invention, installed in a plasma chamber.

FIG. 5A is a perspective view of an embodiment of the present invention for treating large-area workpieces.

FIG. 5B is a cross-sectional view of the embodiment of the present invention shown in FIG. 5A for treating large-area workpieces.

### DETAILED DESCRIPTION OF THE PRESENT INVENTION

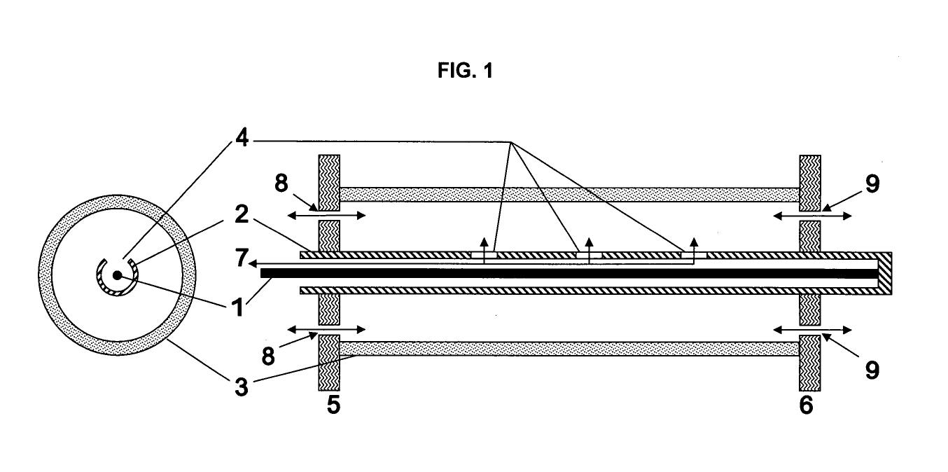

FIG. 1 shows a cross-section and a longitudinal section of a device for generating microwave plasmas, comprising a microwave feed that is configured in the form of a coaxial resonator. Said microwave feed contains an inner conductor (1), an outer conductor (2) and coupling points (4). The microwave feed is surrounded by a dielectric tube (3) which separates the microwave feeding region from the plasma chamber (not shown) and on whose outer side the plasma is formed. The dielectric tube (3) is connected with the walls (5, 6) in a gas-tight or vacuum-tight manner.

A dielectric fluid may be fed or discharged, respectively, via the openings (8) and (9) in the walls. A further possibility for feeding and discharge, respectively, of the dielectric fluid is along the path (7) through the coaxial generator.

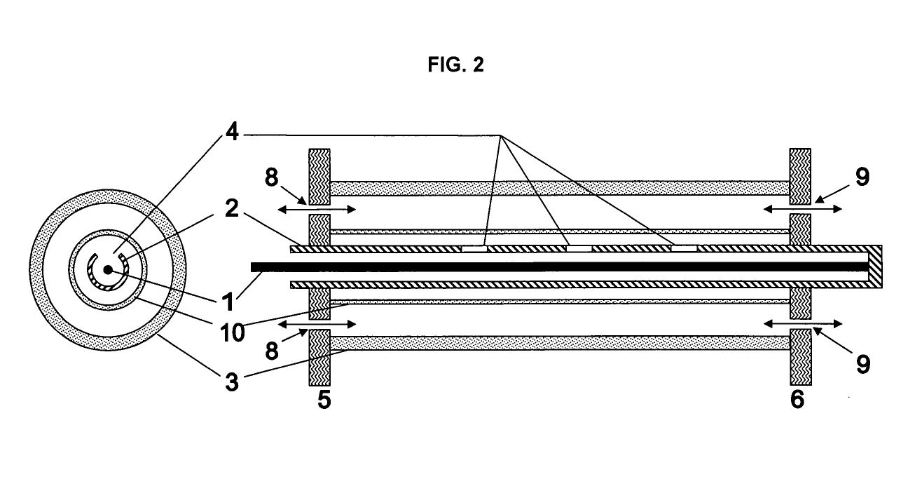

FIG. 2 shows, in a front and side view, a further embodiment of the device, comprising a microwave feed configured as a coaxial resonator, as described in FIG. 1, consisting of the inner conductor (1), the outer conductor (2) and the coupling points (4). The microwave feed is surrounded by a dielectric tube (3) which separates the microwave-feeding region from the plasma chamber (not shown) and on whose outer side the plasma is formed. The dielectric tube (3) is connected with the walls (5, 6) in a gas-tight or vacuum-tight manner. Between the coaxial generator and the dielectric tube (3) there is inserted a dielectric inner tube (10), which is likewise connected with the walls (5, 6) in a gas-tight or vacuum-tight manner. The dielectric fluid is fed or discharged through the space between the dielectric tube (3) and the dielectric inner tube (10), via the openings (8) and (9). By means of this double-tube arrangement it is possible to separate the region through which flows the dielectric fluid, from the microwave feed.

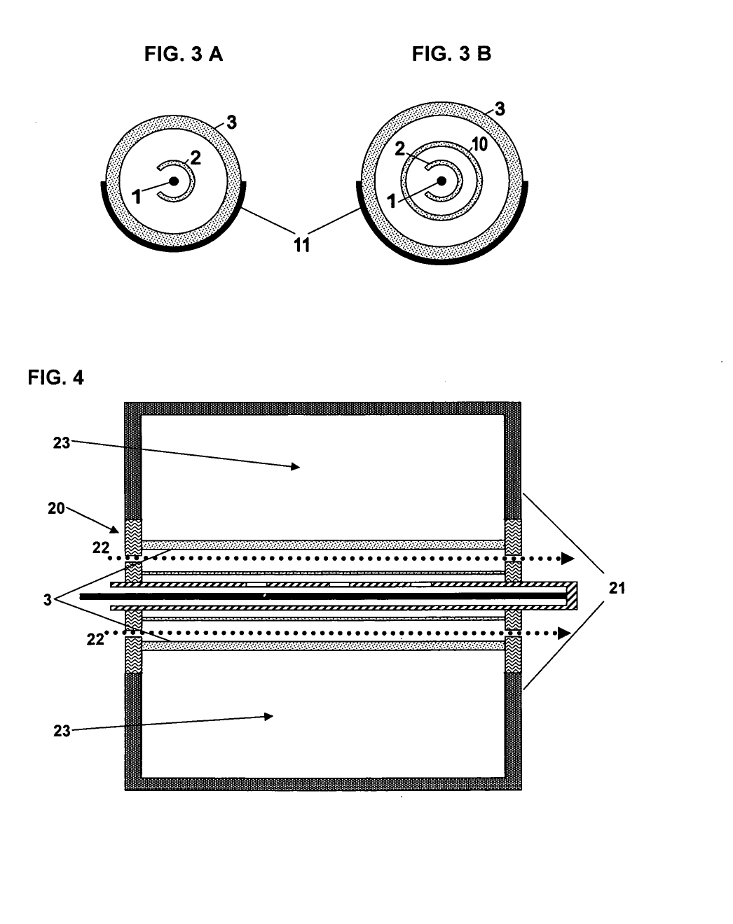

FIGS. 3A and 3B show cross-sections of the embodiments shown in FIGS. 1 and 2, wherein the dielectric tube (3) is surrounded by a metallic jacket (11). What is shown here is the case where the metallic jacket limits the angular range, in which the plasma is formed, to 180°.

FIG. 4 shows a longitudinal section of a device (20), as described in FIG. 1, in a state installed in a plasma chamber (21). The cooling liquid (22) in this example flows through passages in the two end faces. In service, plasma is formed in the space (23) between the outer dielectric tube (3) and the wall of the plasma chamber.

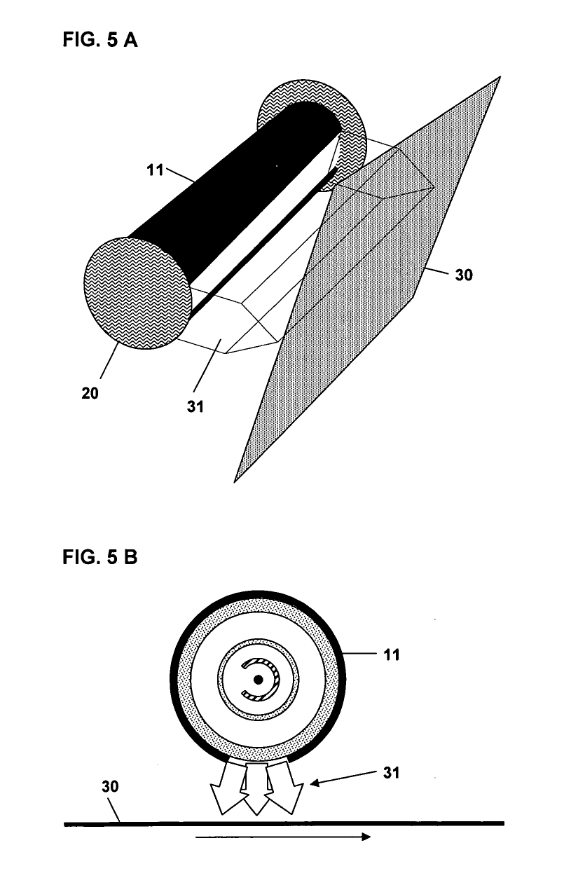

FIGS. 5A and 5B show, in a perspective representation and in a cross-section, an embodiment (20) wherein the major part of the lateral surface of the outer dielectric tube is enclosed by a metal jacket (11) and wherein a plasma (31), which is depicted in the drawing by transparent arrows, can only be formed in a narrow region. In this region, a workpiece (30), moving relative to the device, can be treated with the plasma over a large surface area.

All of the embodiments are fed by a microwave supply, not shown in the drawings, consisting of a microwave generator and, optionally, additional elements. These elements may comprise, for example, circulators, insulators, tuning elements (e.g. three-pin tuner or E/H tuner) as well as mode converters (e.g. rectangular or coaxial conductors).

There are numerous fields of application for the above described device and the above described method. Plasma treatment is employed, for example, for coating, cleaning, modifying and etching of workpieces, for the treatment of medical implants, for the treatment of textiles, for sterilisation, for light generation, preferably in the infrared to ultraviolet spectral region, for conversion of gases or for the synthesis of gases, as well as in gas purification technology. The workpiece or gas to be treated is brought into contact with the plasma or microwave radiation. The geometry of the workpieces to be treated ranges from flat substrates, fibres and webs to shaped articles of any shape.

Due to the increased plasma power, it is possible to achieve higher plasma densities and thereby higher process velocities than with devices and methods according to the prior art.

What has been described above are preferred aspects of the present invention. It is of course not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, combinations, modifications, and variations that fall within the spirit and scope of the appended claims.

I claim:

1. A method for generating microwave plasmas in a device, said device comprising at least one microwave feed that is surrounded by a dielectric tube and having a space between said at least one microwave feed and said dielectric tube, comprising the steps of flowing a dielectric fluid through the space between the at least one microwave feed and the dielectric tube, said dielectric fluid having a low dielectric loss factor tan δ in the range of from 10⁻² to 10⁻⁷.

2. The method according to claim 1, wherein a dielectric inner tube is arranged between the at least one microwave feed and the dielectric tube, said dielectric inner tube surrounding the microwave feed, and said method further comprising the step of flowing the dielectric fluid through the space between the dielectric tube and the dielectric inner tube.

3. The method according to claim 1, wherein each dielectric tube has opposing end faces and is, at the respective end faces, connected with walls having passages, and said method further comprises the step of feeding and discharging the dielectric fluid via said passages in said walls, or via a passage in the at least one microwave feed and at least one of the passages.

4. The method according to claim 2, wherein the dielectric fluid is a dielectric liquid.

5. The method according to claim 4, wherein the dielectric fluid is selected from the group consisting of a mineral oil, a silicone oil and a mixture of both oil groups.

6. The method according to claim 4, wherein the dielectric fluid is a dimethyl polysiloxane.

7. The method according to claim 1, wherein the fluid is or contains a gas.

8. The method according to claim 2, wherein the pressure in the space between the dielectric inner tube and the outer dielectric tube is higher than or equal to atmospheric pressure.

9. The method according to claim 2, wherein the pressure in the space between the dielectric inner tube and the outer dielectric tube is smaller than atmospheric pressure.

10. A device for carrying out the method for generating microwave plasmas according to claim 1, said device comprising at least one microwave feed and at least one dielectric tube for surrounding said at least one microwave feed, each dielectric tube comprising walls for closing each dielectric tube at the respective end faces, wherein at least one of the walls as well as the microwave structure have at least one passage, or that each of the two walls has at least one passage, said at least one passage for conducting a fluid therethrough.

11. The device according to claim 10, wherein said at least one dielectric tube comprise a material selected from the group consisting of metal oxides, semimetal oxides, ceramics, plastics, and composite materials of these substances.

12. The device according to claim 10, further comprising a metal jacket for partially surrounding the outer dielectric tube.

13. The device according to claim 12, wherein said metal jacket comprises one selected from the group consisting of a metallic tube segment, a metal foil and a metal layer.

14. The device according to claim 12, wherein the metal jacket leaves free a region of the lateral surface of the outer dielectric tube, said region extending over the entire length of the dielectric tube.

15. The device according to claim 10, further comprising a process chamber outside the outer dielectric tube.

16. The device according to claim 10, wherein said at least one microwave feed is selected from the group consisting of a microwave antenna and a cavity resonator with coupling points.

17. The device according to claim 10, further comprising microwave feed lines and a microwave generator, said microwave feed lines connecting the at least one microwave feed with said microwave generator.

18. Use of a method according to claim 1 for generating a plasma for coating, cleaning, modifying and etching of workpieces, for treating medical implants, for treating textiles, for sterilisation, for light generation, for light generation in the infrared to ultraviolet spectral region, for converting gases or for gas synthesis, as well as in waste gas purification technology.

19. Use of a device according to claim 10 for generating a plasma for coating, cleaning, modifying and etching of workpieces, for treating medical implants, for treating textiles, for sterilisation, for light generation, for light generation in the infrared to ultraviolet spectral region, for converting gases or for gas synthesis, as well as in waste gas purification technology.

20. The method according to claim 4, wherein said dielectric liquid is an insulating oil.

21. The method according to claim 6, wherein said dimethyl polysiloxane is hexadimethylsiloxane.

22. The device according to claim 11, wherein said at least one dielectric tube comprises a material selected from the group consisting of silica glass and aluminium oxide.

23. The device according to claim 16, wherein said at least one microwave feed is a coaxial resonator.

24. The device according to claim 17, wherein said microwave feed lines are selected from the group consisting of hollow waveguides and coaxial conductors, and wherein said microwave generator is selected from the group consisting of a klystron and a magnetron.
